(19) United States
(12) Patent Application Publication
(10) Pub. No.: US 2009/0162393 A1
(43) Pub. Date: Jun. 25, 2009

(54) Vaccine composition and uses thereof

(75) Inventors: Matt Cook, Campbell (AU); Nick Elliot, Campbell (AU); Jawahar Patil, Campbell (AU); Barbara Nowak, Hobart Tasmania (AU); Michael Attard, Hobart Tasmania (AU); Phil Crosbie, Hobart Tasmania (AU); Richard Morrison, Hobart Tasmania (AU); Giles Campbell, Campbell (AU); Bronwyn Holmes, Campbell (AU); Christopher Prideaux, Campbell (AU)

Correspondence Address: SCULLY SCOTT MURPHY & PRESSER, PC, 400 GARDEN CITY PLAZA, SUITE 300, GARDEN CITY, NY, 11530, US

(73) Assignees: Commonwealth Scientific and Industrial Research Organization, Campbell (AU); Cooperative Research Centre for Sustainable Aquaculture of Finfish (Aquafin CRC), West Beach (AU)

(21) Appl. No.: 11/906,440

(22) Filed: Oct. 1, 2007

(30) Foreign Application Priority Data
Jul. 19, 2007 (AU) 2007903901

(51) Int. Cl. A61K 39/00

(52) U.S. Cl. 424/191.1; 424/265.1

(57) ABSTRACT

The present invention provides a vaccine composition including a nucleic acid that encodes an amoeba-derived antigen (or a peptide molecule encoded thereby), or fragments or variants thereof. The present invention also provides for the use of such vaccine compositions in eliciting an immune response and/or protective immunity in a host against amoebic infection and screening a sample for the presence of amoebae.

The present invention relates generally to a vaccine composition and its use in eliciting an immune response to, and/or protective immunity against, a microbial and/or parasitic infection in an animal, more specifically in eliciting an immune response to, and/or protective immunity against, amoebic infection in aquatic species.

### BACKGROUND

Whilst the vaccine composition according to the present invention may have application in eliciting an immune response to, and/or protective immunity against, a microbial infection in a variety of animals susceptible to such infection, for the purpose of brevity, the following discussion will focus on its application in eliciting an immune response to, and/or protective immunity against, amoebae infection, particularly in fish.

Amoebic gill disease (AGD) is currently considered to be the most significant health problem for farmed Atlantic salmon in Tasmania (Australia), costing the industry an estimated AU$10 million per annum. Associated with extensive morbidity/mortality and reduced production of Atlantic salmon, AGD is caused by the amoeba Neoparamoeba spp that infects the gills of cultured salmon.

Salmon infected with amoebae are safe to eat, but lose condition, experience slower growth and eventually die if untreated. They must be regularly bathed in freshwater to detach the amoebae from their gills. Freshwater bathing has been shown to significantly reduce the number of amoebae on the gills, with an 86±9% reduction in the number of live amoebae found on the gills after freshwater bathing. However, amoeba numbers return to pre-bath levels after only 10 days. Therefore, whilst the results show that commercial freshwater bathing can be effective at reducing amoebae from the gills of fish, reinfection can occur within a week. For this reason, freshwater bathing is not considered a viable, long-term solution. Moreover, the freshwater bathing process calls for additional labour, facilities, and freshwater supplies. In addition, the concentration of calcium and magnesium ions in the water allows the amoeba to survive even very dilute water conditions, allowing them to survive the freshwater bathing process used on salmon farms. As a result, there is the potential for the amoebae, removed by bathing, to re-infect the salmon.

Further studies have also looked at the use of oxidizing chemicals (e.g., chlorine dioxide, chloramine-T and hydrogen peroxide) in artificially hardened freshwater, which have previously been shown to be acutely toxic, at least in respect of isolated amoebae. However, whilst chlorine dioxide and chloramine-T at concentrations of 25 and 10-25 ppm have been shown to reduce the number of gill amoebae by approximately 50% compared with untreated fish, hydrogen peroxide gave variable results with no clear efficacy in terms of removing amoebae from salmon gills. Thus, this approach is still far from favourable, as there is still a considerable number of amoebae remaining in the infected gills of salmon. Moreover, analysis of the fish gills from fish tested with oxidizing chemicals at 50 ppm revealed significant degeneration and necrosis of the gill epithelium indicative of oxidative damage.

Another approach aimed at reducing the number of amoebae that infect the gills of farmed salmon utilizes the movement of water over the gills of the salmon to dislodge the amoebae that sit on the gill surface irritating the gill tissue. However, when affected fish are made to swim steadily for 2 hours at about 1.6 bodylengths per second (1.5 knots), the number of amoebae on the gills remained unchanged, possibly attributed to the respiration rate of the fish not increasing sufficiently to significantly increase water movement over the gills. By contrast, when the fish are towed in a cage from one site to another (over a period of 30 hours at a speed of 1.5 knots), the number of amoebae on the gills of towed fish was reduced. However, the reduction in amoeba numbers was not sufficient from a commercially perspective, nor is it practical to tow a sea cage for 30 hours.

The use of in-feed amoebocides or treatments that help to overcome the effects of AGD has also been investigated, although amoebae such as Neoparamoeba spp. are insufficiently affected by many families of anti-protozoal drugs. The use of mucolytic drugs to enhance mucus sloughing and reduced mucus viscosity has shown promise in retarding the onset of AGD, although their use adds considerable ongoing costs to animal farming.

Thus, there is a need to develop a more effective strategy to overcome the problem associated with microbial infection in animals, especially in regards to AGD in fish such as salmon. The present invention overcomes, or at least alleviates, some of the aforementioned problems of the art by providing a vaccine composition aimed at eliciting an immune response and/or protective immunity against microbial infection in animals, more particularly for eliciting an immune response and/or protective immunity against amoebic infection in fish.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

It is an aspect of the present invention to provide a vaccine composition including a nucleic acid molecule that encodes an antigen, or a fragment or variant thereof, wherein the encoded antigen, or the fragment or variant thereof, is capable of eliciting an immune response and/or protective immunity in an animal against amoebae infection.

In one embodiment, the encoded antigen, or the fragment or variant thereof, has an amino acid sequence that is at least 75% identical, at least 90% identical or at least 95% identical to any one of SEQ ID NOS: 7, 8, 9, 10, 11 or 12, or a fragment or variant thereof. In another embodiment, the encoded antigen, or the fragment or variant thereof, has an amino acid sequence that is selected from the group consisting of SEQ ID NOS: 7, 8, 9, 10, 11 and 12, or a fragment or variant thereof.

The vaccine composition according to the present invention may include any two, three, four or five nucleic acid molecules that each encodes an antigen with an amino acid sequence selected from the group consisting of SEQ ID NOS: 7, 8, 9, 10, 11 and 12, or fragments or variants thereof. In one embodiment, the vaccine composition includes all six nucleic acid molecules, wherein each nucleic acid molecule encodes an antigen with an amino acid sequence as shown in SEQ ID NOS: 7, 8, 9, 10, 11 and 12, or fragments or variants thereof.

In a further embodiment, the vaccine composition includes a nucleic acid molecule, wherein the nucleic acid molecule includes a nucleic acid sequence that is at least 75% identical, at least 90% identical or at least 95% identical to any one of SEQ ID NOS: 1, 2, 3, 4, 5 or 6, or a fragment or variant thereof.

In another embodiment, the vaccine composition includes a nucleic acid molecule, wherein the nucleic acid molecule is selected from the group consisting of SEQ ID NOS: 1, 2, 3, 4, 5 and 6, or a fragment or variant thereof.

The vaccine composition according to the present invention may include any two, three, four or five nucleic acid molecules selected from the group consisting of SEQ ID NOS: 1, 2, 3, 4, 5 and 6, or fragments or variants thereof. In a certain embodiment, the vaccine composition will include all six nucleic acid molecules as shown in SEQ ID NOS: 1, 2, 3, 4, 5 and 6, or fragments or variants thereof.

It is also an aspect of the present invention to provide a vaccine composition including a peptide molecule, or a fragment or variant thereof, that is capable of eliciting an immune response and/or protective immunity in an animal against amoebae infection.

In a certain embodiment of the present invention, the vaccine composition includes a peptide molecule, or a fragment or variant thereof, including an amino acid sequence that is at least 75% identical, at least 90% identical or at least 95% identical to any one of SEQ ID NOS: 7, 8, 9, 10, 11 or 12, or a fragment or variant thereof. In another embodiment of the present invention, the vaccine composition includes a peptide molecule, or a fragment or variant thereof, including an amino acid sequence selected from the group consisting of SEQ ID NOS: 7, 8, 9, 10, 11 and 12.

The vaccine composition according to the present invention may include any two, three, four or five peptide molecules, or fragments or variants thereof, selected from the group consisting of SEQ ID NOS: 7, 8, 9, 10, 11 and 12, or fragments or variants thereof. In another embodiment, the vaccine composition will include all six peptide molecules, or fragments or variants thereof, as shown in SEQ ID NOS: 7, 8, 9, 10, 11 and 12, or fragments or variants thereof.

The present invention also provides for an isolated nucleic acid molecule (or a fragment or variant thereof) that encodes an antigen, or a fragment or variant thereof, wherein the encoded antigen, or the fragment or variant thereof, is capable of eliciting an immune response and/or protective immunity in an animal against amoebae infection, as hereinbefore described.

The present invention also provides an isolated peptide molecule (or a fragment or variant thereof), that is capable of eliciting an immune response and/or protective immunity in an animal against amoebae infection, as hereinbefore described.

It is another aspect of the present invention to provide a method of eliciting an immune response and/or providing protective immunity in an animal against an amoeba infection, the method including administering to the animal a vaccine composition as herein described. In one embodiment, the animal is a fish, such as an Atlantic salmon. In yet another embodiment, the amoebae infection includes, but is not limited to, amoebic gill disease (AGD) attributed to Neoparamoeba spp.

It is also an aspect of the present invention to provide a method of screening a sample for amoebae, the method including the step of detecting a nucleic acid molecule selected from the group consisting of SEQ ID NOS:1 to 6, or a fragment or variant thereof, in the sample. In a further aspect of the present invention, there is provided a method of screening a sample for amoebae, the method including detecting a peptide molecule selected from the group consisting of SEQ ID NOS:7 to 12, or a fragment or variant thereof, in the sample.

The present invention also provides a kit including a container and a vaccine composition as herein described contained therein.

### FIGURES

FIG. 1 shows a nucleic acid sequence (SEQ ID NO:1) derived from Neoparamoeba pemaquidensis encoding a 61 amino acid.

FIG. 2 shows a nucleic acid sequence (SEQ ID NO:2) derived from Neoparamoeba pemaquidensis encoding a 156 amino acid protein.

FIG. 3 shows a nucleic acid sequence (SEQ ID NO:3) derived from Neoparamoeba pemaquidensis encoding a 208 amino acid protein.

FIG. 4 shows a nucleic acid sequence (SEQ ID NO:4) derived from Neoparamoeba pemaquidensis encoding an 89 amino acid protein.

FIG. 5 shows a nucleic acid sequence (SEQ ID NO:5) derived from Neoparamoeba pemaquidensis encoding a 134 amino acid protein.

FIG. 6 shows a nucleic acid sequence (SEQ ID NO:6) derived from Neoparamoeba pemaquidensis encoding a 109 amino acid protein.

FIG. 7 shows the predicted amino acid sequence (SEQ ID NO:7) of the nucleic acid sequence shown in SEQ ID NO:1.

FIG. 8 shows the predicted amino acid sequence (SEQ ID NO:8) of the nucleic acid sequence shown in SEQ ID NO:2.

FIG. 9 shows the predicted amino acid sequence (SEQ ID NO:9) of the nucleic acid sequence shown in SEQ ID NO:3.

FIG. 10 shows the predicted amino acid sequence (SEQ ID NO:10) of the nucleic acid sequence shown in SEQ ID NO:4.

FIG. 11 shows the predicted amino acid sequence (SEQ ID NO:11) of the nucleic acid sequence shown in SEQ ID NO:5.

FIG. 12 shows the predicted amino acid sequence (SEQ ID NO:12) of the nucleic acid sequence shown in SEQ ID NO:6.

FIG. 13 shows a nucleic acid sequence (SEQ ID NO:13) of the Atlantic salmon β-actin promoter.

### DETAILED DESCRIPTION OF THE PRESENT INVENTION

Vaccination is one of the most effective methods for controlling infectious diseases. Vaccines are commercially available for several bacterial pathogens, and considerable research has been conducted on vaccines for both bacterial and viral pathogens of fish. The aim of an effective vaccine is to develop a level of immunity that is equivalent to recovery from natural infection with the agent. However, attempts to control AGD in farmed Atlantic salmon identified a number of difficulties despite considerable research in the area. For instance, earlier reports indicated that immunization with crude, whole parasite preparations does not result in detectable levels of protection from infection, despite the production of antibodies in the serum of treated fish. In the case of AGD, recovery from natural infection was thought not to result in protection from re-infection, suggesting that the development of an effective AGD vaccine was not possible. Thus, surprisingly, applicants provide a vaccine composition capable of eliciting an immune response and/or protective immunity in an animal against amoebic infection.

### Polynucleotide-Based Vaccine Composition

According to a first aspect, the present invention provides a polynucleotide-based vaccine composition including a nucleic acid molecule that encodes an antigen, or a fragment or variant thereof, wherein the encoded antigen, or the fragment or variant thereof, is capable of eliciting an immune response and/or protective immunity in an animal against amoebic infection.

Polynucleotide-based vaccine compositions, when introduced into an animal, will typically induce the expression of the encoded proteins within the animal, causing the animal's immune system to become reactive against the encoded proteins.

The advantage of a polynucleotide-based vaccine composition is that it encodes a defined, often small, number of proteins and, therefore, one of skill in the art can repetitively immunize the animal. Polynucleotide-based vaccine compositions are also advantageous in being relatively easy and inexpensive to manufacture. This method of immunization is similar to the use of viral immunization vectors, but without the additional foreign antigens introduced with a viral vector and, therefore, with less risk of an overwhelming immune response to the vector itself. In addition, the polynucleotide sequences used for immunization may remain within cells at the site of immunization, providing a constant source of antigenic stimulation. Persistent antigen expression may therefore lead to long-lived immunity.

In a certain embodiment of the present invention, the polynucleotide-based vaccine composition of the present invention includes a nucleic acid molecule that encodes an antigen, or a fragment or variant thereof, wherein the encoded antigen, or the fragment or variant thereof, includes an amino acid sequence that is at least 75% identical, at least 90% identical or at least 95% identical to any one of SEQ ID NOS: 7, 8, 9, 10, 11 or 12, or a fragment or variant thereof. In another embodiment, the encoded antigen, or the fragment or variant thereof, has an amino acid sequence that is selected from the group consisting of SEQ ID NOS: 7, 8, 9, 10, 11 and 12, or a fragment or variant thereof.

In yet another embodiment of the present invention, the polynucleotide-based vaccine composition may include any two, three, four or five nucleic acid molecules that each encodes an antigen, or a fragment or variant thereof, wherein the encoded antigens, or the fragments or variants thereof, each include an amino acid sequence selected from the group consisting of SEQ ID NOS: 7, 8, 9, 10, 11 and 12, or fragments or variants thereof. In a certain embodiment, the vaccine composition includes all six nucleic acid molecules, wherein each nucleic acid molecule encodes an antigen with an amino acid sequence as shown in SEQ ID NOS: 7, 8, 9, 10, 11 and 12, or fragments or variants thereof.

In a further embodiment of the present invention, the polynucleotide-based vaccine composition of the present invention includes a nucleic acid molecule selected from the group consisting of SEQ ID NO: 1, 2, 3, 4, 5 and 6, or a fragment or variant thereof.

It will be readily recognizable that the vaccine composition of the present invention may include any nucleic acid molecule that encodes an antigen (or any of its fragments, derivatives, equivalents, variants, mutants etc) capable of eliciting an immune response and/or protective immunity in an animal against any type of amoeba infection, so as to provide, for example, a prophylactic effect against such infection. In one embodiment, the encoded antigen (or any of its fragments, derivatives, equivalents, variants, mutants etc) is capable of eliciting an immune response and/or protective immunity in an animal against amoeba gill disease in fish that is caused, for example, by the amoebae Neoparamoeba spp (e.g. Neoparamoeba perurans).

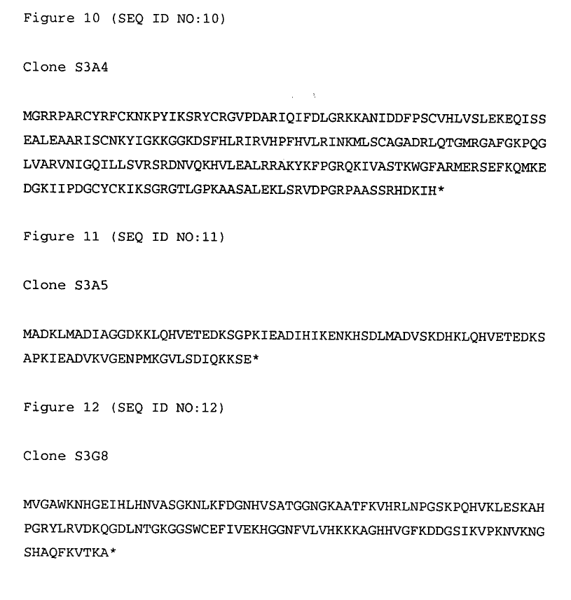

As is well known to those skilled in the art, antigens having substantial amino acid sequence similarities typically cause identical or very similar immune reaction in a host animal. Accordingly, any nucleic acid sequence encoding a derivative, equivalent, variant, fragment, or mutant of any of the amino acid sequences shown in FIGS. 7 to 12 is also suitable for the present invention.

The administration of a polynucleotide-based vaccine composition according to the present invention to an animal (such as Atlantic salmon; Salmo salar) will elicit an immune response to the encoded antigen(s). In a certain embodiment of the present invention, the polynucleotide-based vaccine composition includes one or more nucleic acid molecules, wherein the one or more nucleic acid molecules each include a nucleic acid sequences as shown in SEQ ID NO: 1, 2, 3, 4, 5 and 6 (or fragments or variants thereof). It has also been found that a polynucleotide-based vaccine composition including all six nucleic acid molecules as shown in SEQ ID NOS: 1 to 6 will typically elicit the strongest immune response and/or protective immunity in the animal against amoeba infection.

The antigens encoded by the nucleic acid sequences of the present invention may be functional peptides, but not necessarily so, as long as the peptide encoded by these are sufficiently immunogenic so as to elicit an immune response and/or protective immunity against amoeba infection.

It would also be readily apparent to those ordinarily skilled in the art that fragments, variants or derivatives of the nucleotide sequence of the present invention can be produced which alter the amino acid sequence of the encoded antigen, yet still encode an antigen that is sufficiently immunogenic so as to be capable of eliciting an immune response and/or protective immunity against an amoeba infection. Thus, the altered expressed protein may have an altered amino acid sequence from the native antigen, for example by conservative substitution, yet still elicit an immune response to the native antigen. As used herein, the term “native antigen” typically refers to an antigen that is expressed by an amoeba found in nature.

As used herein, the term “conservative substitution” typically denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids which can be substituted for one another include asparagine, glutamine, serine and threonine. The term “conservative substitution” also includes the use of a substituted amino acid in place of an unsubstituted parent amino acid. As such, it should be understood that in the context of the present invention, a conservative substitution is recognized in the art as a substitution of one amino acid for another amino acid that has similar properties.

In one embodiment of the present invention, the polynucleotide-based vaccine composition includes a nucleic acid molecule, or a fragment, variant or derivative thereof, wherein the nucleic acid molecule, or the fragment, variant or derivative thereof, is at least 75% identical, at least 90% identical or at least 95% identical to any of the nucleic acid sequences shown in SEQ ID NOS: 1 to 6. Percentage sequence identity between nucleotide sequences may be determined either manually by one skilled in the art, or by using computer-based sequence comparison and identification tools that employ algorithms such as BLAST (Basic Local Alignment Search Tool). In yet another embodiment, the vaccine composition includes a nucleic acid molecule selected from the group consisting of SEQ ID NOS: 1, 2, 3, 4, 5 and 6, or fragments or variants thereof.

Fragments of the full-length nucleic acid sequences which encode portions of the full-length antigens (e.g., as depicted in FIGS. 7 to 12) may also be constructed. These fragments may encode an antigen (i.e., a protein or peptide) which is capable of eliciting an immune response and/or protective immunity against the native protein or peptide.

The nucleic acid molecules of the present invention may also include deletions, additions or substitutions of different nucleotide residues resulting in a sequence that encodes the same or substantially similar gene product. The gene product itself may contain deletions, additions or substitutions of amino acid residues which result in a silent change, thus producing a protein with the same or substantially similar immunogenic property. Such amino acid substitutions can be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine, asparagine, glutamine; serine, threonine; phenylalanine, tyrosine.

As used herein, an antigen (i.e., protein or peptide), or a fragment or variant thereof, with an “immunogenic property” typically refers to an antigen, or a fragment or variant thereof, that is capable of being recognized by the immune system of the host animal, but perhaps not necessarily with the same affinity as the native antigen.

The nucleic acid sequences of the present invention can be engineered in order to alter the amoebic protein coding sequence for a variety of ends including, but not limited to, alterations that modify processing and expression of the gene product. For example, mutations can be introduced using techniques that are well known in the art, e.g. by site-directed mutagenesis, to insert new restriction sites, and the like.

In a certain embodiment, the nucleic acid molecule(s) of the polynucleotide-based vaccine composition of the present invention can be ligated into an expression vector which has been specifically optimized for polynucleotide-based vaccinations. Suitable regulatory vectors include any plasmid DNA construct including the nucleic acid molecule(s) of the present invention operatively linked to a promoter. Examples of such vectors include pCI-Xcm-CAT and pbS-Xcm-CAT (The Commonwealth Scientific and Industrial Research Organisation; CSIRO) or commercially available vectors such as p26-DEST, pCDNA3.1 or pVAX (Invitrogen Corporation). The expression vector may also include an initiation codon, a stop codon, and a polyadenylation signal. As is known in the art, these elements are preferably operably linked to the nucleotide sequence(s) that encodes the desired protein and are often selected so as to be operable in the species to which they are to be administered.

In a certain embodiment, the nucleic acid molecule of the present invention is linked to a transcriptional promoter. The use of tissue-specific promoters or enhancers may be desirable to limit expression of the polynucleotide to a particular tissue type.

Initiation codons and stop codons may be included in frame as part of a nucleic acid sequence(s) that encodes an amoeba-derived antigen in the polynucleotide-based vaccine composition according to the present invention.

Promoters and polyadenylation signals included in a polynucleotide-based vaccine composition of the present invention may be selected to be functional within the cells of the animal to be immunized, as are know in the art. Examples of promoters useful in the vaccines of the present invention, especially in the production of a genetic vaccine for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from fish genes, such as β-actin, heat shock protein (HSP) or major histocompatability complex (MHC) promoter elements.

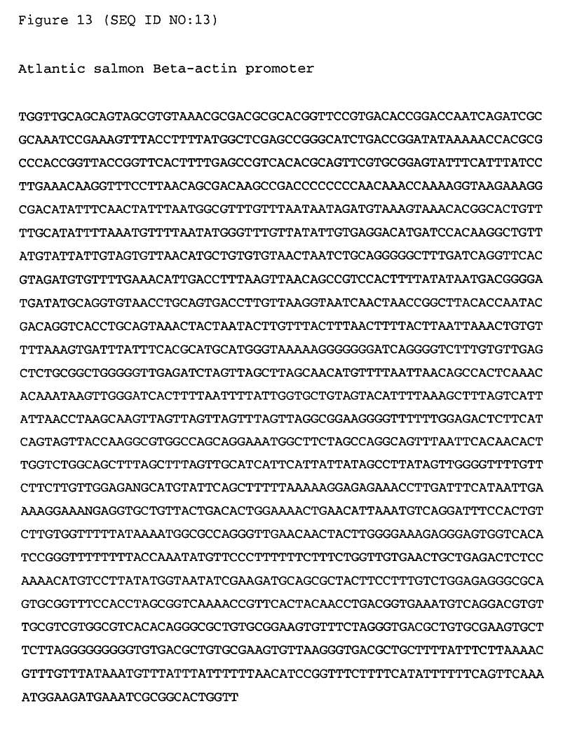

In a certain embodiment, the nucleic acid molecule of the present invention is linked to an Atlantic salmon β-actin promoter, as depicted in FIG. 13 (SEQ ID NO:13).

Examples of polyadenylation signals useful in the vaccine composition of the present invention, especially in the production of a genetic vaccine for aquatic species, include but are not limited to SV40 polyA and BGH polyA.

Nucleic acid molecules useful in the vaccines of the present invention may also include “naked” DNA, as defined, for example, in Restifo et al. (Gene Therapy, 2000, 7:89-92), the disclosure of which is incorporated by reference. Alternatively, the nucleic acid molecule(s) can be operably incorporated in a carrier or delivery vector. Useful delivery vectors include biodegradable microcapsules, immuno-stimulating complexes (ISCOMs) or liposomes, and genetically engineered attenuated live carriers such as viruses or bacteria.

Examples of suitable attenuated live bacterial carriers/delivery vectors include Salmonella typhimurium, Salmonella typhi, Listeria monocytogenes, Shigella, Bacillus, Lactobacillus, Bacille Calmette-Guerin (BCG), Escherichia coli, Vibrio cholerae, Campylobacter, and any other suitable bacterial vector, as is known in the art. Preferred bacterial delivery vectors include attenuated Salmonella typhimurium and attenuated Listeria monocytogenes; particularly preferred is attenuated Salmonella typhimurium. Methods of transforming live bacterial vectors with an exogenous DNA construct are well described in the art (see, for example, Joseph Sambrook and David W. Russell, Molecular Cloning, A Laboratory Manual, 3rd Ed., Cold Spring Harbor Laboratory Press, Cold Spring Harbor, N.Y.; 2001).

In a certain embodiment of the present invention, attenuated viral carriers include herpes viruses, adenoviruses, vaccinia virus, and avipox virus. Methods of transforming a viral vector with an exogenous DNA construct are also well described in the art (see Sambrook and Russell, above).

Liposome carriers are typically unilamellar or multilamellar vesicles, having a membrane portion formed of lipophilic material and an interior aqueous portion. The aqueous portion is used in the present invention to contain the polynucleotide material to be delivered to the target cell. It is generally preferred that the liposome forming materials have a cationic group, such as a quaternary ammonium group, and one or more lipophilic groups, such as saturated or unsaturated alkyl groups having about 6 to about 30 carbon atoms. Certain suitable liposome-forming cationic lipid compounds are described in the literature (see, for example, Stamatatos et al., Biochemistry 27:3917-3925 (1988); and Eibl et al., Biophysical Chemistry 10:261-271 (1979)). Alternatively, a microsphere such as a polylactide-coglycolide biodegradable microsphere may be utilized. A nucleic acid molecule(s) may be encapsulated or otherwise complexed with the liposome or microsphere for delivery of the nucleic acid molecule(s) to a tissue, as is known in the art.

The polynucleotide-based vaccine composition of the present invention can also be administered in conjunction with a facilitating agent that improves the uptake of the genetic material of the vaccine by the cells. In certain embodiments, the nucleic acid molecule(s) can be formulated with or administered in conjunction with a facilitator selected from the group consisting of DNA vaccine adjuvants (e.g., IL-1β and/or CpG's benzoic acid esters, anilides, amidines, urethans and the hydrochloride salts thereof such as those of the family of local anaesthetics, such as disclosed in U.S. Pat. No. 6,248,565, the disclosure of which is incorporated herein by reference.

The vaccine composition according to the present invention may further include a pharmaceutically acceptable carrier.

As used herein, the term “pharmaceutically acceptable carrier” typically means a vehicle for containing the vaccine composition of the present that can be injected into an animal without eliciting an adverse effect. Suitable pharmaceutically acceptable carriers known in the art include, but are not limited to liposomes, gold particles and phosphate buffered saline. Carriers may further include auxiliary agents such as, but not limited to, diluents, stabilizers (i.e., sugars and amino acids), preservatives, wetting agents, emulsifying agents, pH buffering agents, viscosity enhancing additives, colours and the like.

The amount of nucleic acid molecule(s) present in the polynucleotide-based vaccine composition of the present invention is typically a therapeutically effective amount. A therapeutically effective amount of nucleic acid molecule is that amount necessary so that the encoded protein(s) performs its immunological role without causing overly negative effects in the host to which the vaccine composition is administered.

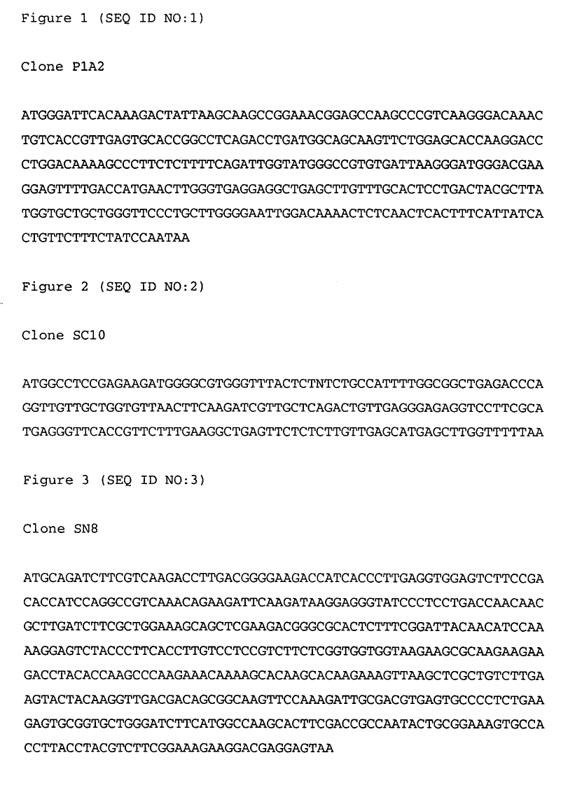

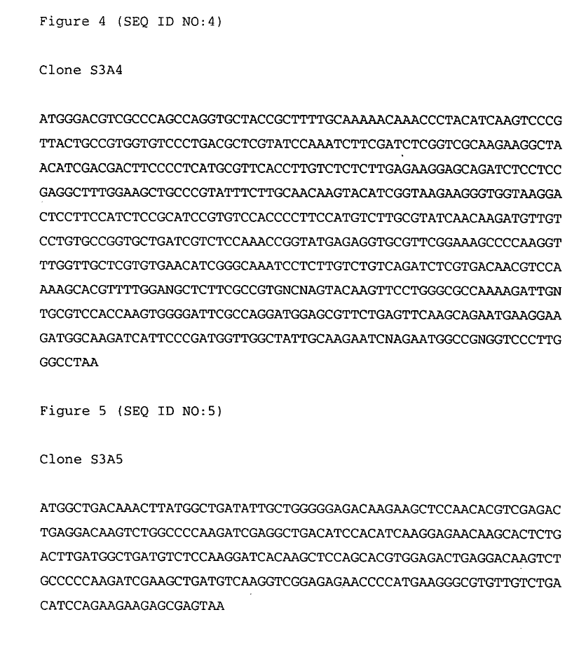

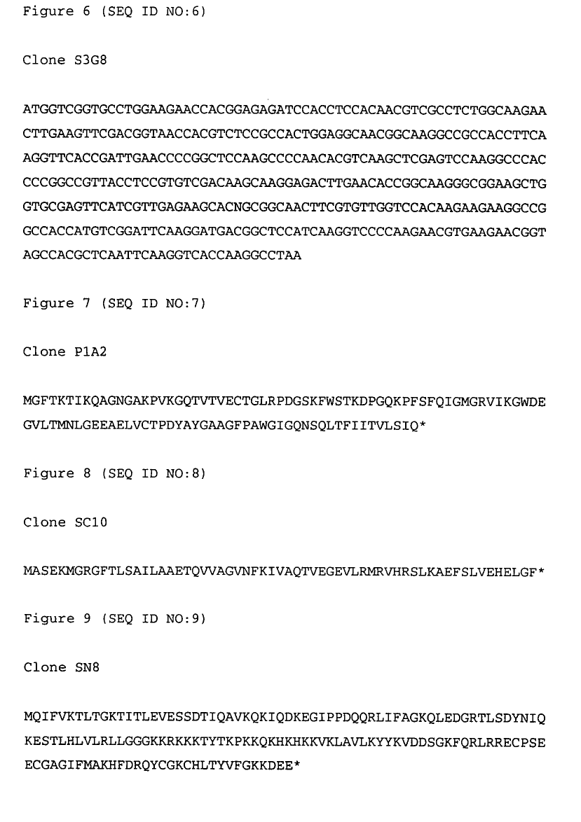

The amount of nucleic acid molecule(s) to be used and the vaccine composition to be administered will vary according to factors such as the strength of the transcriptional and translational promoters used, the extent of existing infection (if any), the mode of administration, as well as the presence of other ingredients in the composition. In one embodiment, the vaccine composition is composed of from about 1 μg to about 2 μg of each of the nucleic acid molecules depicted in FIGS. 1 to 6. The vaccine composition may be administered to the subject on one or more occasions, as necessary to elicit an immune response and/or protective immunity against amoebic infection.

### Peptide-Base Vaccine Composition

It is also an aspect of the present invention to provide a vaccine composition including a peptide molecule, or a fragment or variant thereof, that is capable of eliciting an immune response and/or protective immunity in an animal against amoebic infection.

In one embodiment of the present invention, the vaccine composition may include a peptide molecule, or a fragment or variant thereof, including an amino acid sequence that is at least 75% identical, at least 90% identical or at least 95% identical to any one of SEQ ID NOS: 7, 8, 9, 10, 11 or 12, or a fragment or variant thereof. In a certain embodiment, the vaccine composition includes a peptide molecule, or a fragment or variant thereof, including an amino acid sequence selected from the group consisting of SEQ ID NOS: 7, 8, 9, 10, 11 and 12.

The peptide molecule(s) of the present invention may include an amino acid sequence of a naturally-occurring Neoparamoeba pemaquidensis-derived antigen (or a fragment or variant thereof), such as those selected from the group consisting of SEQ ID NO: 7, 8, 9, 10, 11 and 12. However, it will be readily recognizable that a peptide molecule derived from other species of amoebae, or a derivative, equivalent, variant, mutant etc. thereof, may also be suitable for the instant invention, as long as the peptide molecule is capable of inducing an immune response and/or protective immunity in the host animal against amoeba infection, so as to provide, for example, a prophylactic effect against infection.

In a certain embodiment, the peptide-based vaccine composition includes a peptide that is at least 75% identical, at least 90% identical or at least 95% identical to any of the amino acid sequences shown in SEQ ID NOS: 7 to 12. Percentage sequence identity between amino acid sequences may be determined either manually by one skilled in the art, or by using computer-based sequence comparison and identification tools that employ algorithms such as BLAST (Basic Local Alignment Search Tool).

Whilst the peptide-based vaccine composition may include a peptide molecule, or a fragment or variant thereof, that includes an amino acid sequence selected from the group consisting of SEQ ID NO: 7, 8, 9, 10, 11 and 12 (or fragments or variants thereof), it has been found that a vaccine composition including all six peptide molecules (i.e., with SEQ ID NOS: 7-12) will typically elicit the strongest immune response and/or protective immunity in the animal against an amoeba infection.

As detailed earlier, it would be well recognized by those skilled in the art that peptides having substantial sequence similarities typically cause identical or very similar immune reaction in a host animal. Accordingly, a derivative, equivalent, variant, fragment, or mutant of any of the amino acid sequences shown in FIGS. 7 to 12 may also be suitable for the present invention, as long as the derivative, equivalent, variant, fragment, or mutant is sufficiently immunogenic so as to be capable of eliciting an immune response and/or protective immunity against the amoeba in which the native antigen (i.e., peptide molecule) is expressed. Thus, the altered peptide may have an altered amino acid sequence, for example by conservative substitution (as hereinbefore described), yet still elicit an immune response to the peptide antigen.

The peptide molecule(s) of the present invention can be naturally-derived, or they may be synthesized, for example, by recombinant technologies. Techniques for purifying, synthesizing or producing peptides in recombinant form are well-known in the art and are suitable for production of peptides of sufficient purity for use in the peptide-based vaccine composition of the present invention.

The term “substantially pure”, as used herein, typically denotes a peptide which is substantially free of other compounds with which it may normally be associated in vivo. For example, the term “substantially pure” may refer to homogenous proteins or peptides having an amino acid sequence derived from an amoeba (e.g., SEQ ID NOS:7-12), where homogenicity is determined by reference to purity standards known to those of ordinary skill in the art (such as purity sufficient to allow the N-terminal amino acid sequence of the protein to be obtained). Substantially pure peptides may be obtained from intact microorganisms (particularly bacteria), through microbial expression, by synthesis, and/or by purification means known to those skilled in the art, such as affinity chromatography.

The peptide molecule(s) of the present invention may also be synthesized by such commonly used methods as t-BOC or FMOC protection of alpha-amino groups. Both methods involve stepwise syntheses whereby a single amino acid is added at each step starting from the C terminus of the peptide (see, Coligan, et al., Current Protocols in Immunology, Wiley Interscience, 1991, Unit 9). Peptide molecule(s) of the present invention can also be synthesized by various well known solid phase peptide synthesis methods, such as those described by Merrifield (J. Am. Chem. Soc., 85:2149, 1962), and Stewart and Young (Solid Phase Peptides Synthesis, Freeman, San Francisco, 1969, pp 27 62).

Without being limited by theory, the peptide-based vaccine composition of the present invention typically elicits an immune response and/or protective immunity in an animal by provoking a protective humoral antibody or cell-mediated immune response following its administration to the animal.

### Isolated Nucleic Acid Molecules and Isolated Peptide Molecules

The present invention also provides for an isolated nucleic acid molecule (or a fragment or variant thereof) that encodes an antigen, or a fragment or variant thereof, wherein the encoded antigen, or the fragment or variant thereof, is capable of eliciting an immune response and/or protective immunity in an animal against amoebae infection, as hereinbefore described.

The present invention also provides an isolated peptide molecule (or a fragment or variant thereof), that is capable of eliciting an immune response and/or protective immunity in an animal against amoebae infection, as hereinbefore described.

### Method of Vaccination

It is another aspect of the present invention to provide a method of eliciting an immune response and/or providing protective immunity in an animal against an amoeba infection, the method including administering to the animal a vaccine composition as herein described. In one embodiment, the animal is a fish, such as an Atlantic salmon.

In a certain embodiment, the amoebae infection includes, but is not limited to, amoebic gill disease (AGD) attributed to Neoparamoeba spp.

Where the vaccine composition is a polynucleotide-based vaccine composition, expression of the nucleotide coding sequences in one or more cells in the animal will typically elicit a humoral immune response, a cell-mediated immune response, or both, against the amoeba to which the vaccine composition has been targeted. As used herein, the term “animal” can be any animal susceptible to amoeba infection, including, but not limited to, humans, primates, and aquatic species such as fish (e.g., Atlantic salmon).

As used herein, the phrase “eliciting an immune response” typically refers to a process by which the symptoms of amoebic infection are ameliorated or completely eliminated. As used herein, the phrase “eliciting protective immunity” refers to a process by which the amoebic infection is obstructed or delayed.

In one embodiment, the polynucleotide-based or peptide-based vaccine composition of the present invention is administered to an animal in need of protection against amoebic infection in an amount that is sufficient to elicit an immune response and/or protective immunity against amoeba infection. Without being limited by theory, an immune response elicited by the vaccine composition of the present invention will typically result in reduced numbers of amoeba infecting the animal and minimizing further infection by immunizing the animal against the amoeba.

In a certain embodiment of the present invention, an animal can be sequentially administered a polynucleotide-based vaccine composition, as herein described, in any convenient order, in an amount effective for eliciting an immune response and/or protective immunity against amoeba infection. For instance, an animal may be administered with a vaccine composition including a nucleic acid molecule having a nucleic acid sequence of SEQ ID NO:1, followed by a nucleic acid molecule having a nucleic acid sequence of SEQ ID NO:2, and so on, until a suitable immune response is elicited in the animal so as to protect the animal from infection by diagnostic methods known to those skilled in the art. Alternatively, the animal may be administered with a vaccine composition including any two or more of the six nucleic acid molecules selected from SEQ ID NOS:1 to 6 (or fragments or variants thereof). Similarly, an animal may be administered with a vaccine composition including a peptide molecule having an amino acid sequence of SEQ ID NO:7, followed by a peptide molecule having an amino acid sequence of SEQ ID NO:8, and so on, until a suitable immune response is elicited in the animal so as to protect the animal from infection by diagnostic methods known to those skilled in the art. Alternatively, the animal may be administered with a vaccine composition including any two or more of the six peptide molecules selected from SEQ ID NOS:7 to 12 (or fragments or variants thereof). In yet another embodiment of the present invention, an animal may be administered with a vaccine composition including a nucleic acid molecule encoding an antigen (or a fragment or variant thereof) that includes an amino acid sequence of SEQ ID NO:7, followed by a nucleic acid molecule encoding an antigen peptide molecule (or a fragment or variant thereof) that includes an amino acid sequence of SEQ ID NO:8, and so on, until a suitable immune response is elicited in the animal so as to protect the animal from infection by diagnostic methods known to those skilled in the art. Alternatively, the animal may be administered with a vaccine composition including any two or more of the six nucleic acid molecules encoding an antigen (or a fragment or variant thereof) that includes an amino acid sequence selected from SEQ ID NOS:7 to 12 (or fragments or variants thereof).

In a further embodiment of the present invention, the animal may be administered with any combination of the polynucleotide-based and peptide-based vaccine compositions, as herein described.

In certain embodiments of the present invention, the animal may be first primed with a vaccine composition according to the present invention, followed by a boosting with another vaccine composition comprising the same or a different nucleic acid and/or peptide molecule(s) so as to achieve a robust and long lasting immune response and/or protective immunity against amoebic infection. Both priming and boosting may be by any route of administration, including, but not limited to, intradermal, intramuscular, intravascular, intraperitoneal and oral delivery.

“Naked” plasmid DNA expressing a transgene could also be directly injected intramuscularly, taken up, and expressed. Whilst the efficiency of this approach may be low, with only a small percentage of cells being directly transformed in vivo, and within only a limited area of tissue targeted by this directed delivery, various approaches may be used in conjunction so as to yield a higher efficiency gene delivery method.

In a certain embodiment, the vaccine compositions of the present invention may be used in a conventional prime-boost strategy, in which the same vaccine composition is administered to the animal in multiple doses. In one embodiment, the vaccine composition is used in one or more inoculations. These boosts are performed according to conventional techniques, and can be further optimized empirically in terms of schedule of administration, route of administration, choice of adjuvant and dose.

In one embodiment of the present invention, the vaccine composition can be administered enterally, such as by oral administration, or parenterally, such as by intravenous injection. In another embodiment, the vaccine composition can be administered intramuscularly, intraperitoneally, subcutaneously, intradermally, topically or orally. In certain embodiments, where the vaccine is a polynucleotide-based vaccine composition, the vaccine composition is administered intramuscularly and where the vaccine is a peptide-based vaccine composition, the vaccine composition is administered intraperitoneally.

Typically, though not necessarily, the vaccine compositions of the present invention are provided in a pharmaceutically acceptable carrier, as herein described. For instance, the vaccine compositions of the present invention are formulated with pharmaceutically acceptable carriers and excipients, such as water, saline, dextrose, glycerol, ethanol, and the like, and combinations thereof. The vaccines can also contain auxiliary substances such as wetting agents, emulsifying agents, buffers, and the like.

The vaccine compositions of the present invention may be administered orally to the animal, such as fish, as a solution or suspension in a pharmaceutically acceptable carrier. Where the vaccine composition is a peptide-based vaccine composition, the peptide molecules may be administered in a range from of about 1 microgram to about 100 micrograms per subject. It would also be understood by those skilled in the art that the appropriate dosage will depend upon the subject to be vaccinated, as well as the capacity of the subject's immune system to express the nucleic acids contained in the vaccine composition. The exact dosage chosen may also depend, in part, upon the judgment of the person(s) administering or requesting administration of the vaccine composition.

The effectiveness of the peptide vaccines of this invention may be tested by determining antibody titres to the immunogen, antibody titres to the native protein, and the ability of the antibody to inhibit amoebic infection in a neutralization assay. For example, a peptide-based vaccine composition according to the present invention may be administered subcutaneously in an appropriate adjuvant in groups of four animals with 4, 20, or 100 μg of the peptide-based vaccine. Two to three weeks later, the animals are challenged with a one-half dose of peptide-based vaccine composition emulsified in the appropriate adjuvant. Seven to ten days later, sera is collected and antibody titres to the peptide molecule(s) determined by a standard immunoassay.

The type of amoeba to which the present invention is directed includes, but is not limited to, Neoparamoeba spp.

### Kits

The present invention also provides a kit including a vaccine composition of the present invention packaged in suitable containers such as ampoules, bottles, vials, and the like, either in multi-dose or in unit-dosage forms. The containers may be hermetically sealed after being filled with a vaccine preparation. In one embodiment, the vaccine composition is packaged in a container having a label affixed thereto, which label identifies the vaccine composition and bears a notice in a form prescribed by a relevant regulatory agency reflecting approval of the vaccine composition under appropriate laws, dosage information, and the like. The label preferably contains information about the vaccine composition that is useful to the person(s) administering the vaccine to the subject. The kit may also include printed informational materials relating to the method(s) of administration of the vaccine composition, instructions, indications, and any necessary required warnings.

### Methods of Screening

It is also an aspect of the present invention to provide a method of screening a sample for the presence of amoebae, the method including the step of detecting a nucleic acid molecule selected from the group consisting of SEQ ID NOS:1 to 6, or a fragment or variant thereof, in the sample.

In a further aspect of the present invention, there is provided a method of screening a sample for the presence of amoebae, the method including the step of detecting a peptide molecule selected from the group consisting of SEQ ID NOS:7 to 12, or a fragment or variant thereof, in the sample.

Methods of detecting the presence of amoebae in the sample by detecting a nucleic acid molecule or peptide molecule as herein described would be known to those of ordinary skill in the art. For example, the nucleic acid molecule(s) may be detected by methods such as, but not limited to, Northern blot analysis, reverse transcriptase polymerase chain reaction or in situ hybridization. The peptide molecule(s) may be detected by methods such as, but not limited to, Western blot analysis, immunohistochemistry, SDS-page gel electrophoresis ELISA and immunofluorescence. In a certain embodiment of the present invention, the sample includes, but is not limited to, a tissue sample from a subject suspected of carrying an amoebic infection (e.g., gill tissue from fish) and water from a tank housing an aquatic animal suspected of carrying an amoebic infection.

The present invention will now be illustrated in more detail in the following examples. It is to be understood that these examples serve only to describe the specific embodiments of the present invention, but do not in any way limit the scope of the claims.

Finally, it is to be understood that various other modifications and/or alterations may be made without departing from the spirit of the present invention as outlined herein.

### EXAMPLES

### A. Methodology

### (i) Suppressive Subtractive Hybridization

In order to enrich for transcripts expressed by wild type infective amoebae, Suppressive Subtractive Hybridization (SSH) was performed using a PCR-Select cDNA subtraction kit (BD Biosciences) with WT cDNA as a tester and cultured non-infective cDNA as a driver. The products were PCR amplified after subtraction and ligated into the TA vector pGEM-Teasy (Promega) as per the manufacturer's instructions. The vectors were then used to transform competent E. coli. Transformed cells were then grown overnight on selective media at 37° C. and positive transformants picked into 2 ml LB broth (with ampicillin) and allowed to grow overnight at 37° C. The plasmids were then prepared from culture for sequencing.

### (ii) DNA Sequencing

Plasmid DNA was used as a template for sequencing using a Big-Dye V3.1 cycle sequencing kit. Plasmids were purified using CleanSEQ Dye-terminator removal kit (Agencourt Bioscience Corporation) and sequences were visualised using an ABI 3100 genetic analyser.

(iii) Bioinformatics Analysis

The raw data from DNA sequencing were analysed using ChromasPro V1.22 sequence analysis software. Vector sequences were trimmed automatically and the remaining sequence interrogated. PCR primer sequences and unreliable base calls were edited manually.

The sequence data were then used to interrogate the NCBI (http://www.ncbi.nim.nih.gov/) and the Entamoeba histolytica genome project (http://www.tigr.org/tdb/e2k1/eha1) databases using tblastx (translated nucleotide vs translated protein). Further analysis was performed using a web-based transmembrane prediction program.

### (iv) Expression Library

A full length representational the N. pemaquidensis cDNA library was constructed using the CREATOR SMART cDNA library synthesis kit (BD Biosciences) as per the manufacturers' instructions. The transfer of the N. pemaquidensis cDNA library from pDNR-LIB to pbS-lox was performed using Cre recombinase (BD Biosciences) following the manufacturers' instructions.

This library was used in the expression library immunisation study, comprising 4 pools, each comprising 288 clones, in which 1 pool demonstrated protective clones (as indicated by a 25% increase in protection against amoebic infection following immunization in Atlantic salmon). The protective pool was then subjected to sequencing and interrogated, as detailed above.

### (v) Vaccine Study

A vaccine trial was then conducted, comprising:
Negative control (vector with no gene)
Negative expression control (vector with ‘CAT’ gene)
Putative protective 288 clone pool
Pool of 6 clones plus CAT gene

The pooled selected clones (P1A2, SC10, SN8, S3A4, S3A5, S3G8; each administered at a dose of 6 μg per animal, intramuscularly) offered significant protection against amoeba, demonstrating a 44% increase in relative protection over the negative controls (p<0.05).

### B. β-Actin Promoter

Atlantic salmon genomic DNA was prepared using standard protocols. Genome walking was then used to isolate the promoter with gene specific primers (GSP) designed using the Atlantic salmon β-actin sequence. The DNA was sequenced as indicated above and analyzed for a promoter sequence via the BDGP Neural Network Promoter Prediction Program (http://www.fruitfly.org/seq_tools/promoter.html). Functional analyses were then performed in zebrafish embryos by placing the promoter in pDsRedl-NI.

The β-actin promoter was moved into a pCI-Xcm-CAT vector using standard restriction digest and ligation methods (the new vector was named pbS-Xcm-CAT) and a comparison to the CMV promoter was performed in Atlantic salmon using a standard CAT assay.

1. A vaccine composition including a nucleic acid molecule that encodes an antigen, or a fragment or variant thereof, wherein the encoded antigen, or the fragment or variant thereof, is capable of eliciting an immune response and/or protective immunity in an animal against amoebic infection.

2. The vaccine composition according to claim 1, wherein the encoded antigen, or the fragment or variant thereof, has an amino acid sequence that is at least 75% identical to any one of SEQ ID NOS: 7, 8, 9, 10, 11 or 12.

3. The vaccine composition according to claim 1, wherein the encoded antigen, or the fragment or variant thereof, has an amino acid sequence that is at least 90% identical to any one of SEQ ID NOS: 7, 8, 9, 10, 11 or 12.

4. The vaccine composition according to claim 1, wherein the encoded antigen, or the fragment or variant thereof, has an amino acid sequence that is at least 95% identical to any one of SEQ ID NOS: 7, 8, 9, 10, 11 or 12.

5. The vaccine composition according to claim 1, wherein the encoded antigen, or the fragment or variant thereof, has an amino acid sequence that is selected from the group consisting of SEQ ID NOS: 7, 8, 9, 10, 11 and 12.

6. A vaccine composition including any two nucleic acid molecules that each encodes a peptide molecule including an amino acid sequence selected from the group consisting of SEQ ID NOS: 7, 8, 9, 10, 11 and 12, or fragments or variants thereof.

7. A vaccine composition including any three nucleic acid molecules that each encodes a peptide molecule including an amino acid sequence selected from the group consisting of SEQ ID NOS: 7, 8, 9, 10, 11 and 12, or fragments or variants thereof.

8. A vaccine composition including any four nucleic acid molecules that each encodes a peptide molecule including an amino acid sequence selected from the group consisting of SEQ ID NOS: 7, 8, 9, 10, 11 and 12, or fragments or variants thereof.

9. A vaccine composition including any five nucleic acid molecules that each encodes a peptide molecule including an amino acid sequence selected from the group consisting of SEQ ID NOS: 7, 8, 9, 10, 11 and 12, or fragments or variants thereof.

10. A vaccine composition including nucleic acid molecules that each encodes a peptide molecule including an amino acid sequence as shown in SEQ ID NOS: 7, 8, 9, 10, 11 and 12, or fragments or variants thereof.

11. The vaccine composition according to claim 1, wherein the nucleic acid molecule has a nucleotide sequence that is at least 75% identical to any one of SEQ ID NOS: 1, 2, 3, 4, 5 or 6.

12. The vaccine composition according to claim 1, wherein the nucleic acid molecule has a nucleotide sequence that is at least 90% identical to any one of SEQ ID NOS: 1, 2, 3, 4, 5 or 6.

13. The vaccine composition according to claim 1, wherein the nucleic acid molecule has a nucleotide sequence that is at least 95% identical to any one of SEQ ID NOS: 1, 2, 3, 4, 5 or 6.

14. The vaccine composition according to claim 1, wherein the nucleic acid molecule has a nucleotide sequence that is selected from the group consisting of SEQ ID NOS: 1, 2, 3, 4, 5 and 6.

15. The vaccine composition including any two nucleic acid molecules selected from the group consisting of SEQ ID NOS: 1, 2, 3, 4, 5 and 6, or fragments or variants thereof.

16. The vaccine composition including any three nucleic acid molecules selected from the group consisting of SEQ ID NOS: 1, 2, 3, 4, 5 and 6, or fragments or variants thereof.

17. The vaccine composition including any four nucleic acid molecules sequences selected from the group consisting of SEQ ID NOS: 1, 2, 3, 4, 5 and 6, or fragments or variants thereof.

18. The vaccine composition including any five nucleic acid molecules selected from the group consisting of SEQ ID NOS: 1, 2, 3, 4, 5 and 6, or fragments or variants thereof.

19. The vaccine composition including nucleic acid molecules as shown in SEQ ID NOS: 1, 2, 3, 4, 5 and 6, or fragments or variants thereof.

20. A vaccine composition including a peptide molecule, or a fragment or variant thereof, that is capable of eliciting an immune response and/or protective immunity in an animal against amoebic infection.

21. The vaccine composition according to claim 20, wherein the peptide molecule, or the fragment or variant thereof, includes an amino acid sequence that is at least 75% identical to any one of SEQ ID NOS: 7, 8, 9, 10, 11 or 12.

22. The vaccine composition according to claim 20, wherein the peptide molecule, or the fragment or variant thereof, includes an amino acid sequence that is at least 90% identical to any one of SEQ ID NOS: 7, 8, 9, 10, 11 or 12.

23. The vaccine composition according to claim 20, wherein the peptide molecule, or the fragment or variant thereof, includes an amino acid sequence that is at least 95% identical to any one of SEQ ID NOS: 7, 8, 9, 10, 11 or 12.

24. A vaccine composition including a peptide molecule, or a fragment or variant thereof, wherein the peptide molecule, or the fragment or variant thereof, includes an amino acid sequence selected from the group consisting of SEQ ID NOS: 7, 8, 9, 10, 11 and 12.

25. A vaccine composition including any two peptide molecules selected from the group consisting of SEQ ID NOS: 7, 8, 9, 10, 11 or 12, or fragments or variants thereof.

26. A vaccine composition including any three peptide molecules selected from the group consisting of SEQ ID NOS: 7, 8, 9, 10, 11 or 12, or fragments or variants thereof.

27. A vaccine composition including any four peptide molecules selected from the group consisting of SEQ ID NOS: 7, 8, 9, 10, 11 or 12, or fragments or variants thereof.

28. A vaccine composition including any five peptide molecules selected from the group consisting of SEQ ID NOS: 7, 8, 9, 10, 11 or 12, or fragments or variants thereof.

29. A vaccine composition including peptide molecules each having an amino acid sequence as shown in SEQ ID NOS: 7, 8, 9, 10, 11 and 12, or fragments or variants thereof.

30. A method of eliciting an immune response and/or providing protective immunity in an animal against an amoeba infection, the method including administering to the animal a vaccine composition according to claim 1.

31. The method according to claim 30, wherein the animal is a fish.

32. The method according to claim 31, wherein the fish is an Atlantic salmon.

33. The method according to claim 29, wherein the amoeba is Neoparamoeba spp.

34. A method of screening a sample for amoebae, the method including the step of detecting a nucleic acid molecule selected from the group consisting of SEQ ID NOS:1 to 6, or a fragment or variant thereof, in the sample.

35. A method of screening a sample for amoebae, the method including the step of detecting a peptide molecule selected from the group consisting of SEQ ID NOS:7 to 12, or a fragment or variant thereof, in the sample.

36. A kit including a container and a vaccine composition according to claim 1 contained therein.


SEQUENCE LISTING (12 sequences)

SEQ ID NO: 1 - DNA, 330 bp, Neoparamoeba pemaquidensis
atgggattcacaaagactattaagcaagccggaaacggagccaagcccgtcaagggacaaactgtcaccgttgagtgcaccggcctcagacctgatggcagcaagttctggagcaccaaggaccctggacaaaagcccttctcttttcagattggtatgggccgtgtgattaagggatgggacgaaggagttttgaccatgaacttgggtgaggaggctgagcttgtttgcactcctgactacgcttatggtgctgctgggttccctgcttggggaattggacaaaactctcaactcactttcattatcactgttctttctatccaataa

SEQ ID NO: 2 - DNA, 186 bp, Neoparamoeba pemaquidensis
  misc_feature (36)..(36): n is a, c, g, or t
atggcctccgagaagatggggcgtgggtttactctntctgccattttggcggctgagacccaggttgttgctggtgttaacttcaagatcgttgctcagactgttgagggagaggtccttcgcatgagggttcaccgttctttgaaggctgagttctctcttgttgagcatgagcttggtttttaa

SEQ ID NO: 3 - DNA, 471 bp, Neoparamoeba pemaquidensis
atgcagatcttcgtcaagaccttgacggggaagaccatcacccttgaggtggagtcttccgacaccatccaggccgtcaaacagaagattcaagataaggagggtatccctcctgaccaacaacgcttgatcttcgctggaaagcagctcgaagacgggcgcactctttcggattacaacatccaaaaggagtctacccttcaccttgtcctccgtcttctcggtggtggtaagaagcgcaagaagaagacctacaccaagcccaagaaacaaaagcacaagcacaagaaagttaagctcgctgtcttgaagtactacaaggttgacgacagcggcaagttccaaagattgcgacgtgagtgcccctctgaagagtgcggtgctgggatcttcatggccaagcacttcgaccgccaatactgcggaaagtgccaccttacctacgtcttcggaaagaaggacgaggagtaa

SEQ ID NO: 4 - DNA, 627 bp, Neoparamoeba pemaquidensis
  misc_feature (450)..(450): n is a, c, g, or t
atgggacgtcgcccagccaggtgctaccgcttttgcaaaaacaaaccctacatcaagtcccgttactgccgtggtgtccctgacgctcgtatccaaatcttcgatctcggtcgcaagaaggctaacatcgacgacttcccctcatgcgttcaccttgtctctcttgagaaggagcagatctcctccgaggctttggaagctgcccgtatttcttgcaacaagtacatcggtaagaagggtggtaaggactccttccatctccgcatccgtgtccaccccttccatgtcttgcgtatcaacaagatgttgtcctgtgccggtgctgatcgtctccaaaccggtatgagaggtgcgttcggaaagccccaaggtttggttgctcgtgtgaacatcgggcaaatcctcttgtctgtcagatctcgtgacaacgtccaaaagcacgttttggangctcttcgccgtgncnagtacaagttcctgggcgccaaaagattgntgcgtccaccaagtggggattcgccaggatggagcgttctgagttcaagcagaatgaaggaagatggcaagatcattcccgatggttggctattgcaagaatcnagaatggccgnggtcccttgggcctaa

SEQ ID NO: 5 - DNA, 270 bp, Neoparamoeba pemaquidensis
atggctgacaaacttatggctgatattgctgggggagacaagaagctccaacacgtcgagactgaggacaagtctggccccaagatcgaggctgacatccacatcaaggagaacaagcactctgacttgatggctgatgtctccaaggatcacaagctccagcacgtggagactgaggacaagtctgcccccaagatcgaagctgatgtcaaggtcggagagaaccccatgaagggcgtgttgtctgacatccagaagaagagcgagtaa

SEQ ID NO: 6 - DNA, 405 bp, Neoparamoeba pemaquidensis
  misc_feature (274)..(274): n is a, c, g, or t
atggtcggtgcctggaagaaccacggagagatccacctccacaacgtcgcctctggcaagaacttgaagttcgacggtaaccacgtctccgccactggaggcaacggcaaggccgccaccttcaaggttcaccgattgaaccccggctccaagccccaacacgtcaagctcgagtccaaggcccaccccggccgttacctccgtgtcgacaagcaaggagacttgaacaccggcaagggcggaagctggtgcgagttcatcgttgagaagcacngcggcaacttcgtgttggtccacaagaagaaggccggccaccatgtcggattcaaggatgacggctccatcaaggtccccaagaacgtgaagaacggtagccacgctcaattcaaggtcaccaaggcctaa

SEQ ID NO: 7 - protein, 109 aa, Neoparamoeba pemaquidensis
MGFTKTIKQAGNGAKPVKGQTVTVECTGLRPDGSKFWSTKDPGQKPFSFQIGMGRVIKGWDEGVLTMNLGEEAELVCTPDYAYGAAGFPAWGIGQNSQLTFIITVLSIQ

SEQ ID NO: 8 - protein, 61 aa, Neoparamoeba pemaquidensis
MASEKMGRGFTLSAILAAETQVVAGVNFKIVAQTVEGEVLRMRVHRSLKAEFSLVEHELGF

SEQ ID NO: 9 - protein, 156 aa, Neoparamoeba pemaquidensis
MQIFVKTLTGKTITLEVESSDTIQAVKQKIQDKEGIPPDQQRLIFAGKQLEDGRTLSDYNIQKESTLHLVLRLLGGGKKRKKKTYTKPKKQKHKHKKVKLAVLKYYKVDDSGKFQRLRRECPSEECGAGIFMAKHFDRQYCGKCHLTYVFGKKDEE

SEQ ID NO: 10 - protein, 234 aa, Neoparamoeba pemaquidensis
GRRPARCYRFCKNKPYIKSRYCRGVPDARIQIFDLGRKKANIDDFPSCVHLVSLEKEQISSEALEAARISCNKYIGKKGGKDSFHLRIRVHPFHVLRINKMLSCAGADRLQTGMRGAFGKPQGLVARVNIGQILLSVRSRDNVQKHVLEALRRAKYKFPGRQKIVASTKWGFARMERSEFKQMKEDGKIIPDGCYCKIKSGRGTLGPKAASALEKLSRVDPGRPAASSRHDKIH

SEQ ID NO: 11 - protein, 89 aa, Neoparamoeba pemaquidensis
MADKLMADIAGGDKKLQHVETEDKSGPKIEADIHIKENKHSDLMADVSKDHKLQHVETEDKSAPKIEADVKVGENPMKGVLSDIQKKSE

SEQ ID NO: 12 - protein, 134 aa, Neoparamoeba pemaquidensis
MVGAWKNHGEIHLHNVASGKNLKFDGNHVSATGGNGKAATFKVHRLNPGSKPQHVKLESKAHPGRYLRVDKQGDLNTGKGGSWCEFIVEKHGGNFVLVHKKKAGHHVGFKDDGSIKVPKNVKNGSHAQFKVTKA